(19) United States
(12) Patent Application Publication
(10) Pub. No.: US 2021/0077310 A1
(43) Pub. Date: Mar. 18, 2021

(54) SANITARY PRODUCT WITH INTEGRATED SENSOR, SEMI-MANUFACTURED SANITARY PRODUCT, FABRICATION PLAN THEREFOR, AND METHOD FOR PRODUCING THEREOF

(71) Applicant: Abena Holding A/S, Aabenraa (DK)

(72) Inventor: Per Kammeyer, Rødekro (DK)

(21) Appl. No.: 16/959,909

(22) PCT Filed: Jan. 4, 2019
(86) PCT No.: PCT/DK2019/050003

§ 371 Date: Jul. 2, 2020

(30) Foreign Application Priority Data
Jan. 5, 2018 (DK) PA 201870008

(51) Int. Cl. A61F 13/42; A61F 13/53; A61F 13/15; A61F 13/511; A61F 13/514

(52) U.S. Cl. CPC A61F 13/42; A61F 13/53; A61F 2013/424; A61F 13/511; A61F 13/514; A61F 13/15764

(57) ABSTRACT

Aspects of the present disclosure relate to a sanitary product including a back sheet having an outer surface and an inner surface, a core having a core back side, and a front sheet 106 joined with the back sheet through a joining periphery in a sandwich structure with the core fully positioned within the joining periphery and between the back sheet and the front sheet The inner surface of the back sheet facing the core back side. The sanitary product further including a long direction, a perpendicular short direction and a product periphery including a printed circuit, a stiffener, two through-going incisions and an intersecting cut.

### FIELD OF THE INVENTION

The present invention relates to a sanitary product comprising a back sheet, having an outer surface and an inner surface, a core having a core back side and a front sheet joined with said back sheet through a joining periphery in a sandwich structure with the core fully comprised within the joining periphery. The core is arranged between the back sheet and the front sheet with the inner surface of the back sheet facing the core back side. The sanitary product is configured with a long direction, a perpendicular short direction and a product periphery. The sanitary product comprises a printed circuit comprising a connector configured with a connector width and a sensor for fluid detection, which printed circuit is comprised on a sheet surface facing the core back side. The invention relates to the sanitary product, the fabrication line thereof, the method and a semi-manufactured sanitary product therefore.

### BACKGROUND OF THE INVENTION

A broad variety of sanitary products with incontinence indicators and detection systems exists and are used by people regardless of age from infants to adults. The sanitary products may be absorbent articles and diapers.

Incontinence may have different causes but regardless of the cause for suffering from incontinence, the condition typically involves wearing an absorbent pad or diaper.

For a large group of people suffering from incontinence, checking for wetness may require relying on others. This may be the case for patients in care institutions such as hospitals, and residents in geriatric institutions, care- and nursing homes and similar care centres. An unnoticed wet diaper can become a health risk to the wearer. Diaper rash and skin ulcers are some of the medical conditions that may result from prolonged exposure to wetness.

For both the health risk to the patient and the care providers, processes and procedures for enhanced diaper monitoring are desirable.

To ensure that these patients or residents are adequately cared for, it is necessary for staff to conduct regular checking of the patients suffering from incontinence. The wearers of the sanitary products are often unable to alert staff of the fact that an incontinence event has occurred and, therefore, manual checks are typically carried out on a regular basis. This may place a significant burden on the care providers and causes inconvenience and disruption to the patient.

This need to conduct regular checks of patients for incontinence may be overcome by sanitary products with incontinence indicators and detection systems providing for transmitting a wetness-status of the sanitary product to a remote unit, thereby enabling regular or consistent monitoring of the patient and eliminating the manual checking. The wetness-status may indicate the wetness degree or simply an indicator for whether the fluid is detected or not.

These sanitary products typically include complex sensors with passive and/or active electronic components which may require gentle and careful handling processes which complicate an implementation of a partly or fully automated fabrication process. Furthermore, partly or fully automated fabrication lines may require additional process acts and/or special fabrication units.

Achieving an automated process for incorporation of sensor devices into sanitary products, such as diapers, is complicated by the fact that the sanitary products are generally manufactured at high speed on an assembly line using an in-line process. The inline process typically receives the individual pad layers of the sanitary product as a continuous feed from webs, and applies units in the process comprising rotating drums exerting pressure and tension to the various layers. Furthermore, there is generally little lateral or longitudinal precision involved with the in-line process.

The traditional in-line process or the sensors to be incorporated have to be adjusted either by altering the sensors to be sufficiently robust to withstand the forces exerted to them during the fabrication process by the units comprised in the fabrication line, or by altering the fabrication line to incorporate precision control to ensure the reliability of the integrated sensors to work in view of imprecision and forces exerted on the sensors during the process. Alternatively, the fabrication line is adjusted to exert reduced forces, handle more complex sensor units and work with a higher precision in the assembly of the components comprised in the single sanitary products.

WO2017059831A1 discloses a diaper for electronic detection of body fluids for remote monitoring, wherein the sensor is incorporated as a printed circuit in the diaper.

The printed circuit comprised in the diaper comprises an electrode, to which a transmitter is connected for transmitting the wetness-status of the diaper. However, the electrode is incorporated in the diaper, which means that the transmitter must be connected by conductive pins used to pierce the diaper at the contact part of the electrode. This may require both training and precision in connecting the transmitter to the diaper.

WO 2017/194391A1 also discloses a diaper comprising a wetness detection system. Here two electrodes are bonded to the back sheet or alternatively mechanically anchored to a fabric layer inserted in the diaper. An electronic module can be placed in a pocket included in the diaper and be connected to the two electrodes. The electrodes are mechanically attached to one of the fabric layers of the diaper which may cause stiffness to the diaper and add an additional production step. The electronic module can be connected to the electrodes by sliding the electrodes into the electronics module. However, the fabric layer is only accessible on one fabric layer in a stack of fabric layers which are bonded together in the periphery of the pocket, and thus the accessibility to connecting the electronic module to the electrodes are cumbersome. Attaching the electronic module correctly and to the correct fabric layer may require both training and precision.

### OBJECT OF THE INVENTION

It is an objective to overcome one or more of the before mentioned shortcomings of the prior art. This includes amongst other an objective of providing a sanitary product with an integrated sensor wherein the connection of an electronic module to the integrated sensor is simplified including the accessibility to the connection point. A further objective is to simplify the fabrication of the sanitary product such that the product may be fabricated with high speed in an automated or semi-automated fabrication process.

### DESCRIPTION OF THE INVENTION

An object of the invention may be achieved by a sanitary product comprising a back sheet, having an outer surface and an inner surface, a core having a core back side and a front sheet joined with a joining periphery in a sandwich structure with the core fully comprised within the joining periphery. The core is arranged between the back sheet and the front sheet with the inner surface of the back sheet facing the core back side. The sanitary product is configured with a long direction, a perpendicular short direction and a product periphery. The sanitary product comprises a printed circuit comprising a connector configured with a connector width and a sensor for humidity detection, preferably fluid detection, which printed circuit is comprised on a sheet surface facing the core back side. The sanitary product is further configured with a stiffener for increasing the bending force of the back sheet arranged on the outer surface of the back sheet opposite the connector configured with a stiffener width extending beyond the connector width. The sanitary product is further configured with two through-going incisions in the long direction, between which incisions the connector width is comprised. The incisions are going through at least the stiffener and the back sheet, and an intersecting cut, intersecting the two through-going incisions leaving the connector to be connected to the sensor, such that a flap is created in one or more of the comprised sheets, which flap(s) at least comprise(s) part of the stiffener and the connector. A sanitary product, wherein the joining periphery encircles a smaller area than the product periphery and is comprised within the product periphery, and where the connector, the two through-going incisions in the long direction and the intersecting cut are outside the joining periphery.

The intersecting cut may be the product periphery, whereby the end of the flap is coinciding with the product periphery.

The intersecting cut may be within the product periphery, whereby the end of the flap is comprised outside the joining periphery but not being a part of the product periphery.

The intersecting cut may be a perforation rather than an incision, which may entail that the flap may be fully released once the product is being used.

The sensor for humidity detection is preferably for fluid detection in form of urine. However, it may also be used for detection of humidity in faeces.

The sanitary product may comprise one or more sheets and/or layers in addition to the back sheet, front sheet and the core. An additional sheet with a sheet surface may be comprised between the back sheet and core, where the sheet surface comprises the printed circuit facing the core back side.

One effect of this embodiment may be that the circuit may be printed on a sheet comprising a material, which may have different properties than the back sheet.

This may be advantageous in regard to achieving a sheet material and/or sheet surface which may be more suitable for printing a circuit onto, as the printing techniques and printing inks which may be used may depend on the properties of the sheet material.

A further effect may be that the sheet comprising the printed circuit may be inserted as a separate sheet into the sanitary product independently of the assembly of the remaining product.

This may be advantageous in regard to achieving a more flexible assembly process.

The material chosen for the one or more additional sheets and/or layers may be flexible, so the sanitary product in its entirety is flexible to wear.

The back sheet may comprise a breathable or non-breathable material. The materials may be a textile laminate of polypropylene (PP) and/or polyethylene (PE). The back sheet material may be a nonwoven textile laminate. The back sheet material may be comprised of starch. These are examples of appropriate materials but others may be used.

The back sheet material may have a mass per area in the range 8-80 g/m².

The material chosen for the back sheet may be flexible so that the sanitary product in its entirety is flexible to wear. Furthermore, the back sheet may have a surface making it comfortable to wear against the skin. The sanitary product may not cause any irritations against the skin that can cause rupture, eczema or other skin conditions.

The stiffener may comprise a nonwoven material, polypropylene (PP), polyethylene (PE) and/or other polymer materials which, when applied to the back sheet, increase the bending force of the back sheet. The stiffener may have a mass per area in the range 20-150 g/m².

One effect of the embodiment may be that a sanitary product comprising an integrated sensor is achieved, wherein the risk of misalignment of the integrated sensor during use is eliminated.

A further effect of the embodiment may be that the connector is accessible outside the joint part of the sanitary product. This may be advantageous in regard to not adding a risk of leakage of the sanitary product, when connecting a unit to the connector.

Yet a further effect of the embodiment is that a connector pocket may be formed, comprised by the part of the front sheet and the back sheet outside the joining periphery wherein immediate access to the flap comprising the connector is achieved.

Thus, in immediate continuation hereof, as the stiffener is applied to the back sheet, it may be achieved that the stiffener is not in direct contact with the skin of the wearer of the sanitary product, as the stiffener is arranged such that the part of the front sheet constituting the connector pocket is arranged between the stiffener and the wearers skin when in use.

In a further embodiment of the sanitary product, the sheet surface comprising the printed circuit is the inner surface of the back sheet.

A further effect of this embodiment may be that the integration of the sensor does not add additional individual items to the sanitary product such as additional laminate layers or sensor components, which may be misaligned in the joining of the layers comprised in the product. A further advantage is that the printed circuit on the back sheet provides for a robust sensor design in relation to the standard equipment and processes used in standard fabrication processes. The integrated sensor design thus aids for automated fabrication.

In a further embodiment of the sanitary product, the stiffener is configured with a stiffener width in the short direction in the range 20-35 mm, preferably in the range 25-30 mm, and a stiffener length in the long direction in the range 5-20 mm, preferably in the range 10-15 mm.

Depending on the stiffener material, one effect of this embodiment is that the stiffener is applied to a limited area, thereby only achieving an increased stiffness of the back sheet in a limited area which may be advantageous in regard to not influencing on the wear-ability of the product. This includes not influencing on the comfortability of the product against the skin.

The width of the stiffener depends on the width of the connector. The stiffener may be arranged such that the stiffener extends outside the width of the connector. The length of the stiffener should be sufficient to increase the bending force of the back sheet for an easier handling of the flap for connection purposes.

In another aspect, a stiffening material may be applied to a larger area, extending in length and width, from the size of the stiffener described above, up to the width and/or length of the back sheet. This may have the effect of reinforcing a larger part of the back sheet. This may be advantageous in regard to achieving altered properties of the outer surface of the back sheet and, thereby, the outer side of the sanitary product.

In a further embodiment of the sanitary product, the flap(s) at least comprising the connector and part of the stiffener is adapted for inserting into and connecting to a transmitter unit.

The transmitter unit may comprise transmission means for transmitting a signal to remote means providing monitoring of the wetness-status of the sanitary product. The transmitter unit may transmit a signal to be processed further by the remote means. Alternatively, the transmitter unit may comprise processing means for processing information from the sensor before transmitting information to the remote means.

One effect of this embodiment is that an electrical connection may be provided for, between the connector and the transmitter unit, as the connector of the printed circuit is exposed in the surface of the flap. This may be advantageous in regard to a stable connection. A further effect of this embodiment is that the insertion of the flap in the transmitter unit is made easier by reinforcing the back sheet with the stiffener, thereby achieving an effortless and easy handling when connecting the transmitter unit to the sanitary product. The transmitter unit may comprise a slot for the connector to be inserted into. The width of the flap comprising the connector may be adapted accordingly, such that the width of the flap is below the width of the slot, such that the flap may be displaced within the slot. In other words, a slack is provided for the connector in the slot. The displacement may, however, be limited to a distance, which ensures that the connector and the transmitter units are correctly connected. This distance or slack is therefore determined by the connector pins of the two units and/or the distance between the individual connector pins. Furthermore, the slack should be sufficient for easy insertion of the flap into the transmitter unit but limited such that the position of the flap in the transmitter unit is not causing doubt for the user in regard to obtaining a correct connection.

An object of the invention may be achieved by a semi-manufactured sanitary product comprising a back sheet, having an outer surface and an inner surface, a core having a core back side and a front sheet joined with a joining periphery in a sandwich structure with the core fully comprised within the joining periphery. The core is arranged between the back sheet and the front sheet with the inner surface of the back sheet facing the core back side. The semi-manufactured sanitary product is configured with a long direction, a perpendicular short direction and a product periphery. The semi-manufactured sanitary product comprises a printed circuit comprising a connector configured with a connector width and a sensor for humidity detection, preferably fluid detection, which printed circuit is comprised on a sheet surface facing the core back side, wherein the joining periphery encircles a smaller area than the product periphery and is comprised within the product periphery and where the connector is outside the joining periphery.

One effect of this embodiment is that an automated fabrication process may be achieved for sanitary products with incorporated sensors. The fabrication process may be achieved using standard units already used for in-line production in other industries.

Using a semi-manufactured sanitary product with an incorporated sensor in the form of a printed circuit on a sheet, already comprised in semi-manufactured sanitary product before feeding it into the fabrication line, provides for a robust sensor design in relation to the standard equipment and processes used in standard fabrication processes. The integrated sensor design thus aids for automated fabrication.

In a further embodiment of the semi-manufactured sanitary product, the sheet surface comprising the printed circuit is the inner surface of the back sheet.

A further effect of this embodiment may be that the integration of the sensor does not add additional individual items to the sanitary product such as additional laminate layers or sensor components which may be misaligned in the joining of the layers comprised in the product.

An object of the invention may be achieved by a fabrication line for sanitary products with a production direction adapted for receiving a feed of semi-manufactured sanitary products comprising a product conveyor band for supporting the semi-manufactured sanitary product, and one or more fixtures for holding the semi-manufactured sanitary products The fabrication line further comprises a stiffener applicator configured for applying a stiffener to the back sheet for increasing the bending force of the back sheet on the outer surface opposite the connector configured with a stiffener width extending beyond the connector width. The fabrication line further comprises an incision device configured for applying through-going incisions in at least the long direction on either side of the connector, the incisions going through at least the stiffener and the back sheet, and intersecting an intersecting cut, such that a flap is created in the back sheet comprising part of the stiffener, and which flap further comprises the connector.

In one embodiment of the fabrication line the fixtures may be comprised in the product conveyor.

In one embodiment of the fabrication line the product conveyor may be a linear product conveyor or a carousel product conveyor. The carousel product conveyor may be a turning table.

Alternatively, an object of the invention may be achieved by a fabrication line for sanitary products with a production direction adapted for receiving a feed of semi-manufactured sanitary products comprising a product conveyor band for supporting the semi-manufactured sanitary product, and a fixture conveyor belt configured with fixtures for holding the semi-manufactured sanitary products, which may be coupled to the product conveyor band. The fabrication line further comprises a stiffener applicator configured for applying a stiffener to the back sheet for increasing the bending force of the back sheet on the outer surface opposite the connector configured with a stiffener width extending beyond the connector width. The fabrication line further comprises an incision device configured for applying through-going incisions in at least the long direction on either side of the connector, the incisions going through at least the stiffener and the back sheet, and intersecting an intersecting cut, such that a flap is created in the back sheet comprising part of the stiffener, and which flap further comprises the connector.

In one embodiment of the fabrication line the fixture conveyor is a linear fixture conveyor in case the product conveyor is a linear product conveyor or a carousel fixture conveyor in case the product conveyor is a carousel product conveyor.

The intersecting cut may be the product periphery, whereby the end of the flap is coinciding with the product periphery.

The intersecting cut may be within the product periphery, whereby the end of the flap is comprised outside the joining periphery but not being a part of the product periphery.

The intersecting cut may be a perforation rather than an incision, which may entail that the flap may be fully released once the product is being used.

One effect of these embodiments is that an automated fabrication process may be achieved for sanitary products with incorporated sensors. The fabrication process may be achieved using standard units already used for in-line production in other industries.

Furthermore, the devices comprised in the fabrication line are not adjusted to exert reduced forces, handle more complex sensor units or work with a higher precision in the assembly of the components comprised in the single sanitary products compared to common used devices.

Using a semi-manufactured sanitary product with an incorporated sensor in the form of a printed circuit on a sheet, already comprised in semi-manufactured sanitary product before feeding it into the fabrication line, provides for a robust sensor design in relation to the standard equipment and processes used in standard fabrication processes. The integrated sensor design thus aids for automated fabrication.

A further effect of this embodiment may be that the fabrication line may handle different sizes of semi-manufactured sanitary products. This provides for a fabrication line adaptable for an automated in-line fabrication of small volume product series.

In one embodiment of the fabrication line, the incision device may comprise mechanical cutting means cutting through at least the back sheet with the applied stiffener. This may also include pressure means for pressing the back sheet with applied stiffener element against the mechanical cutting means.

One effect of this embodiment is that a standard incision device may be incorporated in the fabrication line. This device may be advantageous in regard to mechanical stability and providing for a production speed comparable to those of existing fabrication lines for sanitary products. The cutting edges experience wear and should be maintained to ensure sharp incisions. The wear may be highly influenced by the material used for the back sheets and the stiffener.

In a further embodiment of the fabrication line, the incision device comprises a laser.

The incision device may comprise a single laser emitting a beam to be split into two beams for laser cutting through at least the back sheet with the applied stiffener. The incision device may alternatively comprise two lasers, each emitting a single beam. As a further alternative, the incision device may comprise a plurality of lasers for providing specific ranges of wavelengths and effects adjusted to the materials and thickness of the materials of the back sheet and the stiffener.

The incision device may comprise a continuous wave laser, a pulsed laser or a combination.

One effect of this embodiment is that the laser(s) does/do not comprise mechanical parts which experiences wear by physical interaction with the back sheet and the stiffener. Thereby, it will achieve uniform sharpness of the incisions performed in the individual products.

In one aspect, the laser may be a CO₂ laser operating with wavelengths in the spectrum from 9.2-11.4 μm with emitted power in the range of 200-500 W. Alternatively, the laser may be the CO₂ laser operated with emitted power in a range from 200 W up till 4.5 kW.

The wavelength and the emitted power of the laser should be adjusted with respect to the materials to be laser cut to achieve a through-going incision without burning any of the materials.

In a further embodiment the fabrication line further comprises an unfolding device for unfolding a semi-manufactured sanitary product, which unfolding device is adapted to receive a semi-manufactured sanitary product fed into the fabrication line.

The semi-manufactured sanitary products to be fed into the fabrication line may be in the form of folded products. In one aspect, the semi-manufactured sanitary products may be fabricated on commonly used fabrication lines for sanitary products, not comprising sensors. These fabrication lines may output the sanitary products as folded products and thus, similar thereto, output the semi-manufactured sanitary products.

This embodiment may have the effect that the semi-manufactured sanitary products may be fed into the fabrication line without additional manual work when unfolding the products. This may be advantageous, both in regard to reduced costs of the production, but also a reduced risk of misalignment, due to a varying degree of unfolding of the product before feeding it into the fixtures comprised in the fabrication line holding the semi-manufactured sanitary products.

In a further embodiment the fabrication line further comprises automated infeed means for infeed of semi-manufactured sanitary products.

As previously described, the semi-manufactured sanitary products to be fed into the fabrication line may be in the form of folded products. In one aspect, the semi-manufactured sanitary products may be fabricated on common used fabrication lines for sanitary products not comprising sensors. These fabrication lines may output the sanitary products as folded products and thus similar thereto output the semi-manufactured sanitary products.

The infeed means may in one aspect comprise a cassette wherein the semi-manufactured sanitary products may be stack.

This embodiment may have the effect that the semi-manufactured sanitary products may be stack and automatic fed into the fabrication line without additional manual work. This may be advantageous in regard to less manual handling for optimized production speed.

Alternatively, the feeding of the individual semi-manufactured sanitary products may be performed manually. The effects and advantages of a manual feeding are in line with those previously described, namely that a robust sensor design may be fed into an automated fabrication process, where the fabrication process may be achieved using standard units already used for in-line production in other industries. A further effect of this embodiment may be that the fabrication line may handle different sizes of semi-manufactured sanitary products. This provides for a fabrication line adaptable for a manually fed automated in-line fabrication of small volume product series.

An object of the invention may be achieved by a method for producing a sanitary product comprising acts of providing a semi-manufactured sanitary product and, applying a stiffener to the back sheet for increasing the bending force of the back sheet on the outer surface opposite the connector configured with a stiffener width extending beyond the connector width. The method further comprises acts of performing two through-going incisions in at least the long direction of the product on either side of the connector going through at least the stiffener and the back sheet and intersecting an intersecting cut, such that a flap is created in at least the back sheet comprising part of the stiffener, and which flap further comprises the connector.

The intersecting cut may be the product periphery, whereby the end of the flap is coinciding with the product periphery.

The intersecting cut may be within the product periphery, whereby the end of the flap is comprised outside the joining periphery but not being a part of the product periphery.

The intersecting cut may be a perforation rather than an incision, which may entail that the flap may be fully released once the product is being used.

One effect of this method may be that the integration of the sensor only requires acts which are commonly known acts from fabrication methods in other industries, but acts which may be performed in an automated fabrication method and in-line productions. This may be advantageous in regard to achieving a fabrication method which does not require any intermediate acts where any intermediate products are on hold for cure, drying or comparable tasks.

The effects and advantages of the method are in line with those already described in connection with the embodiments of the sanitary product, the fabrication line and the semi-manufactured sanitary product.

Thus, the further effects and advantages are amongst others as listed below.

A further effect of the method is that a connector pocket may be formed, comprised by part of the front sheet and the back sheet outside the joining periphery, wherein immediate access to the flap comprising the connector is achieved.

Thus, in immediate continuation hereof, as the stiffener is applied to the back sheet it may be achieved that the stiffener is not in direct contact with the skin of the wearer of the sanitary product, as the stiffener is arranged in a way where the part of the front sheet constituting the connector pocket is arranged between the stiffener and the user's skin when in use.

The method further incorporates a number of precautionary conditions for mitigating the risk of misalignment and/or imprecision of the sensor position in the product during fabrication.

These precautionary conditions include the stiffener and the two through-going incisions. These may all extend in length in the long direction of the product, which is also the production direction. They may extend in length beyond the product periphery in the end comprising the flap. Furthermore, the stiffener has a width extending beyond the connector width. These conditions in combination ensure that a flap comprising the connector may be achieved, which is reinforced in the entire area comprised in the flap. This may, as previously described, be advantageous in regard to achieving:
a stable connection between the connector and the transmitter unit
easy insertion of the flap in the transmitter unit
easy handling for connecting the transmitter unit to the sanitary product.

An object of the invention may be achieved by a sanitary product obtained by the method for producing a sanitary product.

The effects and advantages of the method are in line with those already described in connection with the embodiments of the sanitary product, the fabrication line, the fabrication method and the semi-manufactured sanitary product.

The following is a step-by-step description of one embodiment of the fabrication process from unfolding semi-manufactured sanitary products, to fixation of the product, applying stiffener to the back sheet and subsequent cutting, including alignment of the product.

The fabrication line may be built up with one or more infeed systems comprising associated product elevators.

Stacks of semi-manufactured sanitary products are manually inserted into the infeed system(s) and placed in the product elevator. The bottom of the elevator can move vertically, as the semi-manufactured sanitary products are pushed out one by one. The fabrication line is equipped with two lifts, ensuring that the line does not stand still when an elevator needs to be filled up. Alternatively, one elevator may be used which can be quickly filled up.

The semi-manufactured sanitary products are received by an unfolding device, which uses suction cups for applying vacuum to the top layer of the semi-manufactured sanitary product. Hereafter, the assembly comprising the suction cups is tilted so that the outer surface of the sanitary product is lifted from the product conveyor.

After the semi-manufactured sanitary product has been lifted, a cylinder is actuated which laterally pushes the suction cups apart such that a part of the outer surface of the semi-manufactured sanitary product is extended and exposed.

With the aid of compressed air, the end of the semi-manufactured sanitary product, onto which the stiffener is to be applied, is unfolded. The fixture comprises an air cylinder which extends an arm of the fixture and simultaneously closes a gripper comprised in the fixture by means of an air cylinder. The purpose of the gripper is to capture and expose the outer layer of the sanitary product, where the mounting of stiffener and cutting is to occur.

A vision system may be used to detect the location of the connector comprised in the semi-manufactured sanitary product. The stiffener applicator and laser are controlled accordingly, so that the stiffener is placed on top of the connector and the cutting takes place on either side of the connector.

In one aspect the laser is stationary and the laser beams may be moved by use of rotational or moving mirrors.

The sanitary products may now be applied with the stiffener and incisions. Afterwards, the sanitary products may be folded and stacked.

In one aspect the fabrication line for sanitary products may comprise a number of stations positioned in the production direction.

The stations may communicate with each other.

The stations may include one station for the stiffener applicator and one station for the incision device.

Further stations may include an infeed station adapted for receiving a semi-manufactured sanitary product. The semi-manufactured sanitary products may be arranged in fixtures either manually or automatically.

Further stations may include one or more vision system. The vision systems may be used for detection of the position of the connector in a coordinate system, for control of the performed tasks or for adjustment of the position of the devices of the individual stations. Adjustments of the position of the devices of the individual stations may be based on the previous detected coordinates of the position of the connector in each of the products feed into the fabrication line.

Further stations may include a rejection station. The rejection station may receive information from the vision system of a detected faulty product and the product may therefore be rejected from the fabrication line instead of continuing to the subsequent stations. The rejection station may comprise a push and release mechanism.

Further stations may include a test station. The test station may be adopted to test the sensor by connecting probes to the connector and measure on an applied electrical signal. This test may be performed after applying the stiffener and the incisions.

Further stations may include an ejection station. An ejection station may comprise a push and release mechanism.

In one aspect the fabrication line may comprise a stacking line.

In one aspect the ejection station may be connected to a chute for transferring the sanitary product to a stacking line or alternatively to a rejection line for the cases where the product fails the test at the test station.

### DESCRIPTION OF THE DRAWING

FIG. 1 illustrates one embodiment of the sanitary product with indications of the core and printed circuit.

FIG. 2 illustrates one embodiment of the sanitary product inserting into and connecting to a transmitter unit.

FIG. 3 illustrates one embodiment of the fabrication line 300 in perspective view.

FIG. 4 illustrates one embodiment of the fabrication line 300 seen from a top view.

FIG. 5 illustrates one embodiment of the fabrication line 300 in perspective view.

FIG. 6 illustrates one embodiment of a chute with connected rejection and stacking lines.

FIG. 7 illustrates one embodiment of the incision device.

FIG. 8 illustrates one embodiment of the incision device.

FIG. 9 illustrates one embodiment of the method for producing a sanitary product.

Detailed Description of the Invention

| No | Item |
|---|---|
| 10 | transmitter unit |
| 20 | connector pocket |
| 100 | sanitary product |
| 102 | back sheet |
| 104 | core |
| 104A | core back side |
| 105 | sheet surface |
| 106 | front sheet |
| 108 | joining periphery |
| 110 | long direction |
| 112 | short direction |
| 114 | product periphery |
| 120 | inner surface |
| 122 | printed circuit |
| 124 | connector |
| 126 | connector width |
| 128 | sensor |
| 130 | outer surface |
| 132 | stiffener |
| 136 | stiffener width |
| 138 | stiffener length |
| 140 | through-going incision |
| 144 | intersecting cut |
| 150 | flap |
| 200 | semi-manufactured sanitary product |
| 300 | fabrication line |
| 302 | production direction |
| 303 | product conveyor |
| 303A | linear product conveyor |
| 303B | carousel product conveyor |
| 304 | fixture conveyor |
| 304A | linear fixture conveyor |
| 304B | carousel fixture conveyor |
| 306 | fixtures |
| 310 | stiffener applicator |
| 320 | incision device |
| 326 | laser |
| 328 | mechanical cutting device |
| 329 | cutting edge |
| 370 | unfolding device |
| 380 | automated infeed means |
| 400 | method |
| 410 | providing |
| 420 | applying |
| 430 | performing |

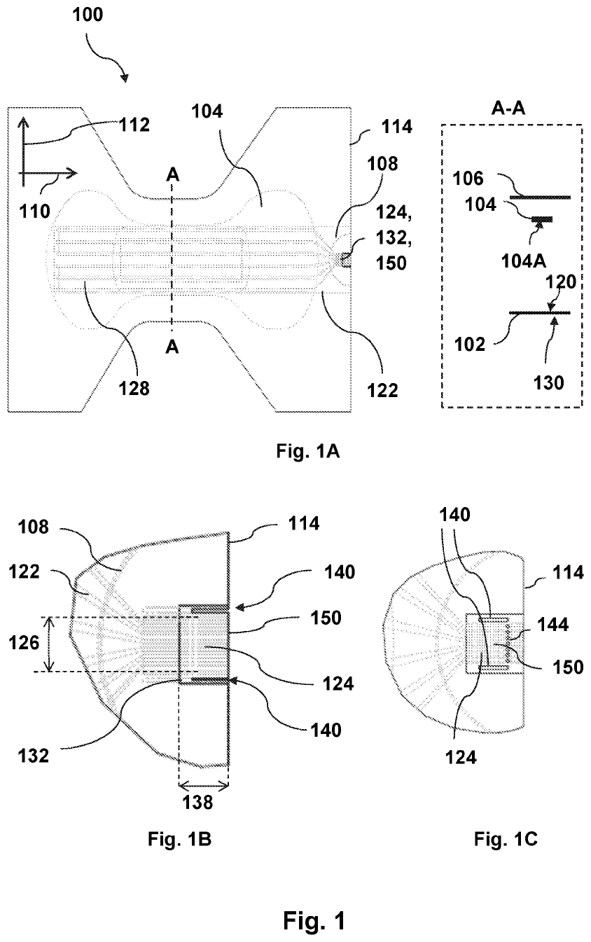

FIG. 1 illustrates one embodiment of the sanitary product 100 with indications of the core 104 and printed circuit 122. FIG. 1A illustrates the entire product 100 and FIG. 1B illustrates a detailed section of the product 100 of the part comprising the connector 124.

The illustrated sanitary product 100 in FIG. 1A comprises a core 104 having a core back side 104A, comprised between a back sheet 102 and a front sheet 106, which is illustrated by the incision A-A shown in the insert A-A). A printed circuit 122 is arranged on a sheet surface 105 facing the core back side. The sheet surface 105 may be the inner surface 120 of the back sheet facing the core back side 104A. The printed circuit 122 comprises a sensor 128 for fluid detection and a connector 124. The back sheet, the core 104 and the front sheet are joined with a joining periphery 108 within which the core 104 is fully comprised. The sanitary product 100 may defined by a long direction 110 and a perpendicular short direction 112. The sanitary product 100 may be further defined by a product periphery 114, being the outer periphery of the product 100. The joining periphery 108 encircles a smaller area than the product periphery 108, which is illustrated in the right hand side of FIG. 1A. Here, the joining periphery 108 is illustrated with a curvature extending inwards towards the core 104 leaving the part of the product comprising the connector 124, the stiffener 132 and the incisions 140 in the stiffener 132 and the back sheet outside of the joining periphery 108. This part is illustrated in more detail in FIG. 1B. The part of the product comprised outside the joining periphery 108 and within the product periphery 114 provides for a pocket (connector pocket 20, not illustrated here) in the product constituted of the front sheet and the back sheet.

The insert A-A illustrates the sanitary product 100 seen from the side in the incision A-A. The illustrated embodiment comprises a core 104 having a core back side 104A comprised between a back sheet 102 and a front sheet 106. In this embodiment the sheet surface 105 facing the core back side 104 is the back sheet 102, where the printed circuit 122 is arranged on the inner surface 120.

A part of the printed circuit 122 comprising the connector 124 is illustrated in FIGS. 1B and 1C. The stiffener, 132 having a stiffener length 138, is arranged opposite the connector 124 on the back sheet and extends in width (in the short direction) beyond the connector width 126.

In FIG. 1B two through-going incisions 140 going through the back sheet and the stiffener 132 is provided on either side of the connector 124 ending at the product periphery 114, thereby, providing for a flap to be enabled in the back sheet. The flap comprising the connector is reinforced such that the bending force of the back sheet is increased.

The two through-going incisions 140 may penetrate through all the sheets comprised in the product between the back sheet and the front sheet, including these two.

The stiffener 132 is preferably arranged so it extends in length in one end to reach the product periphery 114 in the end comprising the non-joined area comprising the connector 124.

In FIG. 1C the two through-going incisions 140 going through the back sheet and the stiffener 132 are also provided on either side of the connector 124, however, the incisions 140 do not extend to the product periphery 114. Instead an intersecting cut 144, is here illustrated as a perforation that intersects the two through-going incisions 140 leaving the connector to be connected to the sensor, so that the flap 150 is created away from the product periphery 114.

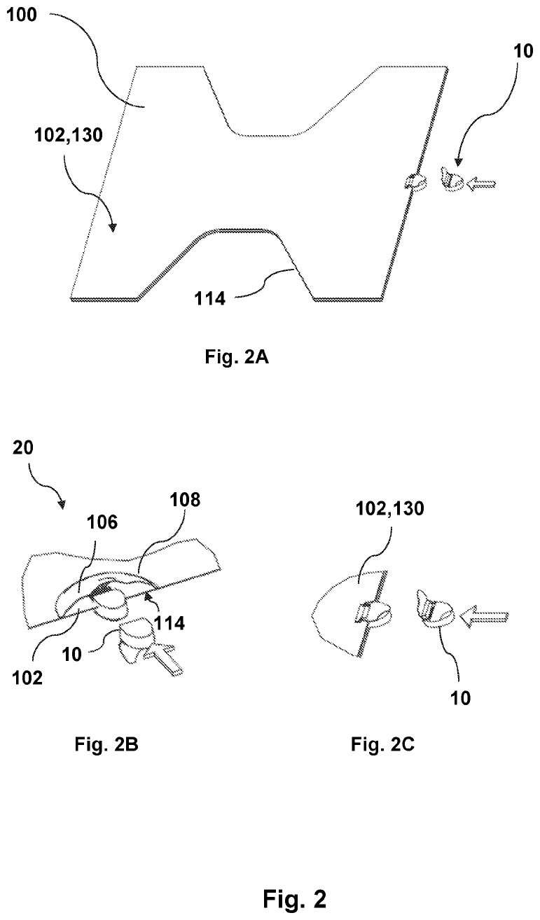

FIG. 2 illustrates one embodiment of the sanitary product 100 where a part of the product is inserted into and connected to a transmitter unit 10 at the product periphery 114. FIG. 2A illustrates the full sanitary product 100 as seen from the outer surface 130 of the back sheet 102. The transmitter unit 10 is connected to the reinforced part of the back sheet 102 comprising the connector.

FIGS. 2B and 2C illustrates the connection of the transmitter unit 10 with the back sheet 102 in more detail, where FIG. 2B illustrates the connection as seen from the top sheet 106, which is the skin side of the sanitary product 100. FIG. 2C illustrates the connection as seen from the outer surface 130 of the back sheet 102, which is the outer side of the sanitary product 100. FIG. 2B further illustrates the connector pocket 20 in the product 100 constituted of the front sheet 106 and the back sheet 102, which connector pocket 20 arises from the part of the product 100 comprised outside the joining periphery 108 and within the product periphery 114.

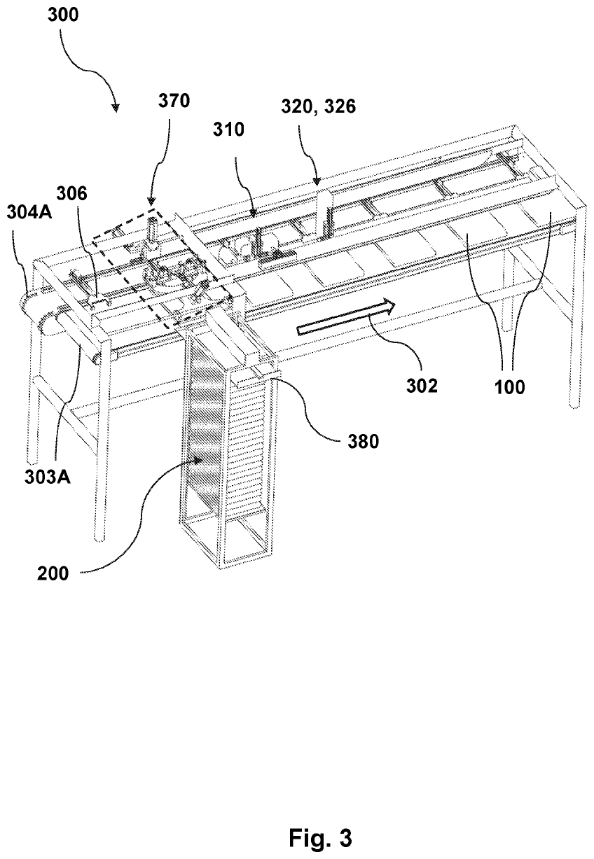

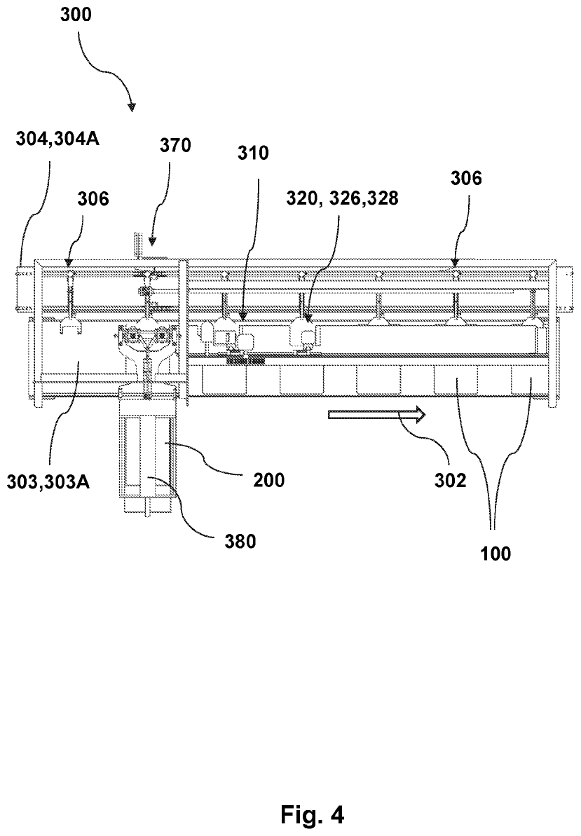

FIGS. 3 and 4 illustrate one embodiment of the fabrication line 300 for sanitary products with a production direction 302. The fabrication line 300 is illustrated in perspective view in FIG. 3 and from a top view in FIG. 4. The fabrication line 300 provides for in-line production with an infeed of semi-manufactured sanitary products 200.

The fabrication line 300 comprises a product conveyor 303, here being a linear product conveyor 303A for supporting the semi-manufactured sanitary product 200 through the line and a fixture conveyor 304, here being a linear fixture conveyor 304A configured with fixtures 306 for holding the semi-manufactured sanitary products 200. The fixture conveyor 304,304A may be coupled to- and decoupled from the product conveyor 303,303A, such that the product may be stopped at a given position where the product conveyer belt may continue to run or vice versa.

The illustrated embodiment comprises a stiffener applicator 310 configured for applying a stiffener to the back sheet on the outer surface for increasing the bending force of the back sheet opposite the connector. The embodiment further comprises an incision device 320 configured for applying through-going incisions in the product in the long direction on either side of the connector. The incisions are performed to go through at least the stiffener, the back sheet and the sheet comprising the printed circuit, which may be the back sheet. The incision device 320 may be a laser 326 or a mechanical cutting device 328 with cutting edges. Such mechanical device may comprise active cutting means and passive pressure means, in between which, the part of the product to which the incisions shall be applied is suspended.

The illustrated embodiment further comprises an unfolding device 370 for unfolding a semi-manufactured sanitary product 200. The unfolding device 370 is adapted to receive a semi-manufactured sanitary product 200 fed into the fabrication line 300.

The illustrated embodiment further comprises automated infeed means 380 for infeed of semi-manufactured sanitary products.

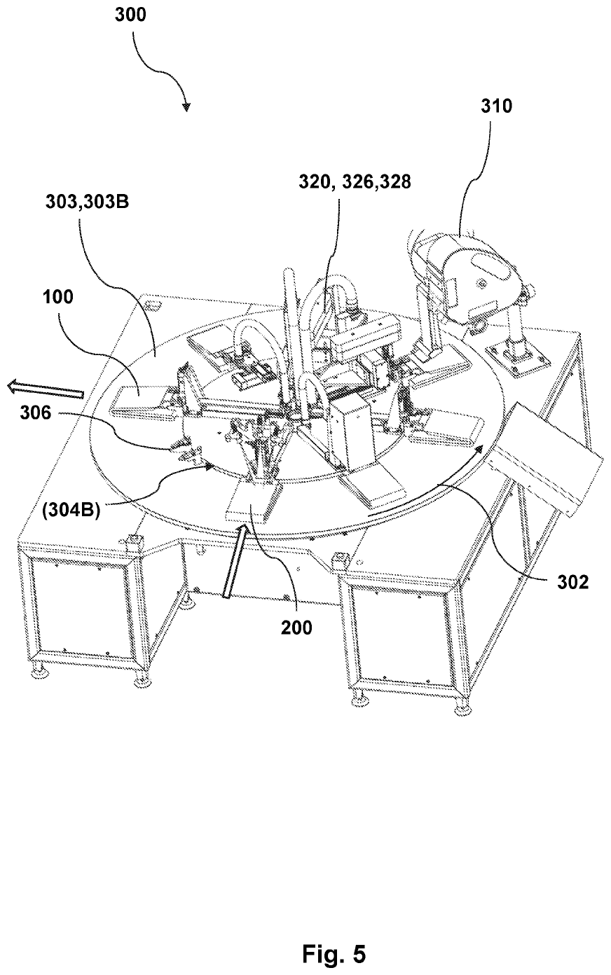

FIG. 5 illustrates one embodiment of the fabrication line 300 for sanitary products with a production direction 302. The fabrication line 300 is illustrated in perspective view. The fabrication line 300 provides for in-line production with an infeed of semi-manufactured sanitary products 200. Arrows indicate infeed of the semi-manufactured sanitary product 200 and ejection of the finished sanitary product 100, respectively.

The fabrication line 300 comprises a carousel product conveyor 303B for supporting the semi-manufactured sanitary product 200 through the fabrication line. The carousel product conveyor 303B is configured with fixtures 306 for holding the semi-manufactured sanitary products 200. Alternatively, the fixtures may be comprised in a separate carousel fixture conveyor 304B which may be coupled to and decoupled from the product conveyor 303,303B, such that the product may be stopped at a given position where the product conveyer 303,303B may continue to run or vice versa. The embodiment with a separate carousel fixture conveyor 304B is not explicitly illustrated in FIG. 5 but one position of the carousel fixture conveyor 304B is indicated with the reference number 304B.

The illustrated embodiment comprises a stiffener applicator 310 configured for applying a stiffener to the back sheet opposite the connector for increasing the bending force of the back sheet. The embodiment further comprises an incision device 320 configured for applying through-going incisions in the product in the long direction on either side of the connector. The incisions are performed to go through at least the stiffener, the back sheet and the sheet comprising the printed circuit, which may be the back sheet. The incision device 320 may be a laser 326 or a mechanical cutting device 328 with cutting edges. Such mechanical device may comprise active cutting means and passive pressure means, in between which, the parts of the product to which the incisions shall be applied is suspended.

The illustrated embodiment of the fabrication line comprises a number of stations positioned in the production direction 302. These stations include one station for the stiffener applicator 310 and one station for the incision device 320.

The stations may communicate with each other.

Further stations may include an infeed station adapted for receiving a semi-manufactured sanitary product 200. Here the semi-manufactured sanitary product 200 may be arranged in fixtures 306 either manually or automatically.

Additional stations may include one or more vision systems for detection of the position of the connector in a coordinate system, for control of the performed tasks or for adjustment of the position of the devices of the individual stations. The position of the devices of the individual stations may be adjusted based on previously detected coordinates of the position of the connector in each of the infeed products. The adjustment of the devices may include tilting, rotation and/or linear dislocation of the devices according to the coordinate system.

Alternatively, the fixtures may be adjustable and include tilting, rotation and/or linear dislocation of the product according to the coordinate system.

The additional stations may include a rejection station. The rejection station may receive information from the vision system if no connector part is detected in a product 200. This product 200 may therefore be rejected from the fabrication line 300 instead of continuing on in the fabrication line to any subsequent stations. The rejection station may comprise a push and release mechanism.

The additional stations may include a test station. The test station may be adopted to test the sensor by connecting probes to the connector and measure on an applied electrical signal. This test may be performed after applying the stiffener and the incisions. The test station may be placed just before the product is ejected from the fabrication line 300. The product may be ejected for stacking or for rejection depending on the result of the performed test. The product may be ejected at an ejection station. The ejection station may comprise a push and release mechanism. The ejection station may be connected to a chute for transferring the resulting sanitary product 100 to a stacking line or alternatively to a rejection line in the cases, where the product fails the test at the test station.

The production direction 302 is illustrated as being counter clockwise. However, the direction 302 and the order of the stations may be changed or interchanged to achieve the most optimal production flow.

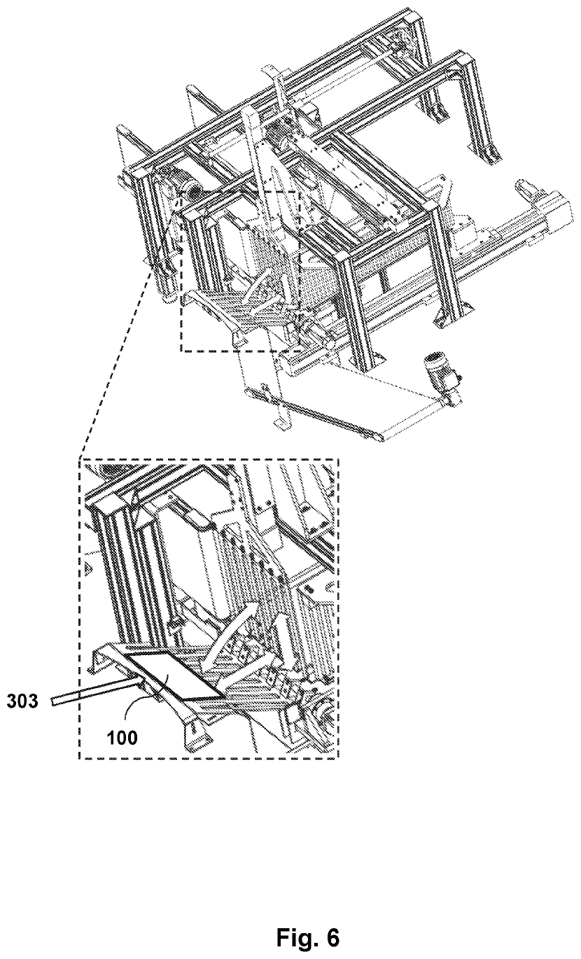

FIG. 6 illustrates one embodiment of a chute with connected rejection line and stacking line. The chute may be adapted for multiple functions. The illustrated chute is constructed with bars and may be operated to establish a connection to a product conveyor e.g. the product conveyor 303,303A,303B of the embodiments illustrated in FIG. 3-5, such that an ejected finished product, here the finished sanitary product 100 is arranged on the chute. The insert illustrated with dotted lines are a close-up of the chute and the surrounding arrangement for stacking the products 100.

The transfer of the sanitary product 100 from the conveyor 303 to the chute is illustrated by an arrow. The chute may be arranged in different positions creating a connection to a stacking line or to a rejection line. The given position may be determined by a communicated status from a test station in the fabrication line 300.

In case the product is to be transferred to the rejection line, the chute may be arranged in a position with an open-end connection, whereby the product 100 slides off the chute into a waste area/rejection line.

In case the product is to be transferred to the stacking line, the chute may be arranged in a position with a closed-end connection, where the product 100 slides to the lower end of the chute connected to the stacking line. The illustrated stacking line comprises a vertical moveable bar-lattice and a stacking area with a horizontal moveable stacking wall. The stacked products in the stacking area are held between the vertical moveable bar-lattice and the horizontal moveable stacking wall.

The chute may be pivoted to an upright position, such that the ejected product 100 is arranged in a vertical position. In the vertical position the product 100 may be arranged between the chute and the vertical moveable bar-lattice. Once the product 100 is in this position, the vertical moveable bar-lattice may be lifted, such that the product 100 may be transferred to the stacking area by a horizontal movement of the chute. The vertical moveable bar-lattice may then be lowered to a position where the bar-lattice is arranged in between the bars of the chute.

Hereafter the chute may be pivoted back to the transfer position.

The product 100 is now held by the vertical moveable bar-lattice may be stacked up against previously stacked products or the horizontal moveable stacking wall in case the product 100 is the first to be stacked.

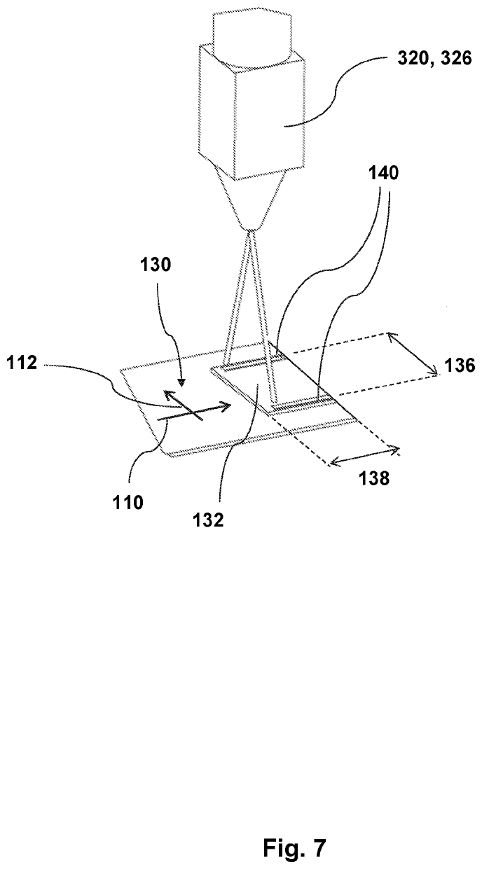

FIG. 7 illustrates one embodiment of the incision device 320. The incision device comprises a laser 326 which is emitting two beams to the back sheet with the applied stiffener 132. The stiffener 132 with a stiffener length 138 and a stiffener width 136 is applied to the outer surface 130 of the back sheet. Two through-going incisions 140 are provided in the long direction 110 of the product, so the connector width is comprised between the incisions 140 in the short direction 112. The incisions are through-going incisions 140, penetrating at least the stiffener 132 and the back sheet.

In one aspect, the incisions may be limited to be within the length of the stiffener 314 to avoid any burning of the back sheet if the laser beams are applied outside the stiffener 132. Such burning may arise because the effect emitted by the laser is adjusted to penetrate a combination of materials, with a thickness larger than the thickness of the back sheet material alone.

In the illustrated embodiment, the incisions are provided in the direction from the stiffener 132 to the back sheet with the inner surface 120 of the back sheet facing away from the laser 326. In an alternative embodiment, the incision may be provided in the opposite direction from the back sheet to the stiffener 132 with the inner surface 120 of the back sheet facing towards the laser 326.

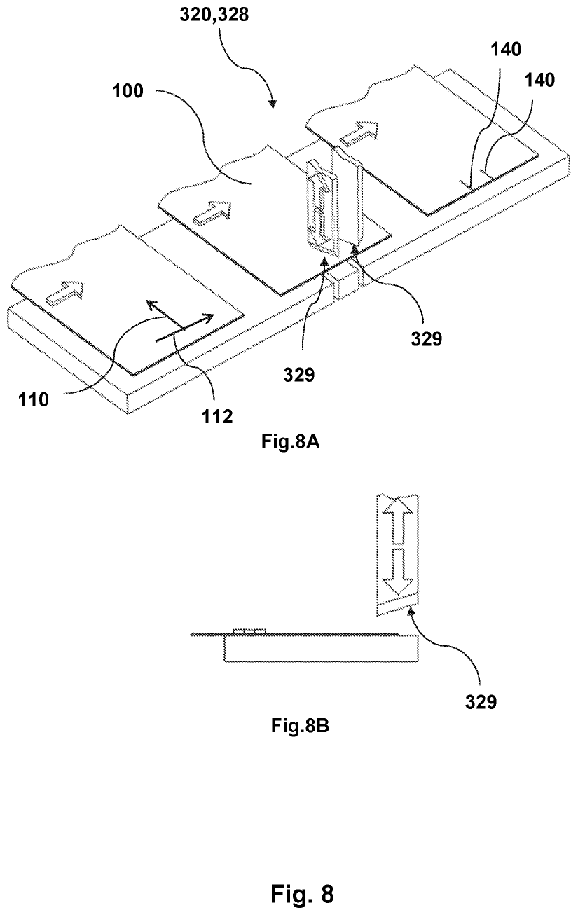

FIG. 8 illustrates another embodiment of the incision device 320. The incision device is illustrated in perspective view in FIG. 8A and in a side view in FIG. 8B. The incision device comprises a mechanical cutting device 328 having two cutting edges 329 for applying the through-going incisions 140 in at least the back sheet with the applied stiffener. The two through-going incisions 140 are provided in the long direction 110 of the product so the connector width is comprised between the incisions 140 in the short direction 112.

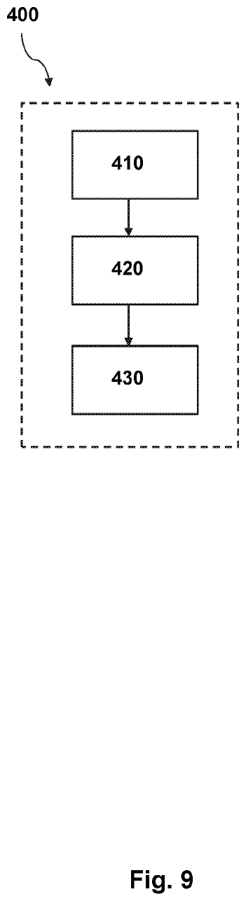

FIG. 9 illustrates one embodiment of the method 400 for producing a sanitary product. The illustrated embodiment comprises the act of providing a semi-manufactured sanitary product. The method further comprises an act of applying 420 a stiffener to the back sheet on the outer surface for increasing the bending force of the back sheet.

The method further comprises an act of performing 430 two through-going incisions in the long direction of the product on either side of the connector, penetrating at least the stiffener and the back sheet and intersecting the product periphery such that a flap is created in at least the back sheet comprising part of the stiffener, and which flap further comprises the connector.

What is claimed is:

1. A sanitary product comprising;
a back sheet having an outer surface and an inner surface;
a core having a core back side;
a front sheet joined with said back sheet through a joining periphery in a sandwich structure with the core fully positioned within the joining periphery and between the back sheet and the front sheet, the inner surface of the back sheet facing the core back side;
a long direction;
a perpendicular short direction;
a product periphery including;
a printed circuit including a connector with a connector width and a sensor configured and arranged for humidity detection, the printed circuit positioned on a sheet surface facing the core back side;
a stiffener configured and arranged for increasing the bending force on the outer surface of the back sheet opposite the connector, and the connector having a stiffener width extending beyond the connector width;
two through-going incisions in the long direction between which the connector width is defined, the incisions going through at least the stiffener and the back sheet; and
an intersecting cut intersecting the two through-going incisions and configured and arranged to leave the connector connected to the sensor, and form a flap in one or more of the sheets, the flap including at least part of the stiffener and the connector,
wherein the joining periphery encircles a smaller area than the product periphery, the joining periphery is within the product periphery, and the connector, the two through-going incisions in the long direction and the intersecting cut are outside the joining periphery.

2. The sanitary product according to claim 1, wherein the sheet surface is the inner surface of the back sheet.

3. The sanitary product according to claim 1; wherein the stiffener includes a stiffener width, in the short direction, in the range 20-35 mm and a stiffener length, in the long direction, in the range 5-20 mm.

4. The sanitary product according to claim 1, wherein the flap is configured and arranged for inserting into and connecting to a transmitter unit.

5. A semi-manufactured sanitary product comprising;
a back sheet having an outer surface and an inner surface;
a core having a core back side;
a front sheet joined with a joining periphery in a sandwich structure with the core fully positioned within the joining periphery and between the back sheet and the front sheet, the inner surface of the back sheet facing the core back side;
a long direction;
a perpendicular short direction;
a product periphery; and
a printed circuit including a connector having a connector width and a sensor configured and arranged for humidity detection, the printed circuit positioned on a sheet surface facing the core back side;
wherein the joining periphery encircles a smaller area than the product periphery and is positioned within the product periphery and where the connector is outside the joining periphery.

6. The semi-manufactured sanitary product according to claim 5, wherein the sheet surface is the inner surface of the back sheet.

7. A system for manufacturing sanitary products with a production direction, the system comprising:
a fabrication line configured and arranged for receiving a feed of semi-manufactured sanitary products
a product conveyor configured and arranged for supporting the semi-manufactured sanitary product;
one or more fixtures configured and arranged for holding the semi-manufactured sanitary products;
a stiffener applicator configured and arranged for applying a stiffener to a back sheet of the semi-manufactured sanitary products for increasing the bending force of the back sheet on an outer surface opposite a connector with a stiffener width extending beyond a connector width of the connector; and
an incision device configured and arranged for applying through-going incisions in at least a long direction on either side of the connector, the incisions going through at least the stiffener and the back sheet and intersecting an intersecting cut, such that a flap is created in the back sheet and encompassing part of the stiffener and the connector.

8. The system of claim 7, wherein the one or more fixtures are positioned within the product conveyor.

9. The system of claim 7, wherein the product conveyor is a linear product conveyor or a carousel product conveyor.

10. The system of claim 7, further including a fixture conveyor configured with the one or more fixtures.

11. The system of claim 10, wherein the fixture conveyor is a linear fixture conveyor where the product conveyor is a linear product conveyor, or a carousel fixture conveyor where the product conveyor is a carousel product conveyor.

12. The system of claim 7, wherein the incision device is a laser.

13. The system of claim 7, further including an unfolding device configured and arranged to receive a semi-manufactured sanitary product fed into the fabrication line and to unfold the semi-manufactured sanitary product.

14. The system of claim 7, further including automated infeed means configured and arranged for infeeding of semi-manufactured sanitary products.

15. A method for producing a sanitary product comprising the steps of:
Providing a semi-manufactured sanitary product;
Applying a stiffener to a back sheet of the sanitary product for increasing the bending force of the back sheet on an outer surface opposite a connector having a stiffener width extending beyond the connector width; and
performing two through-going incisions in at least a long direction of the sanitary product on either side of the connector going through at least the stiffener and the back sheet and intersecting an intersecting cut, such that a flap is created in at least the back sheet, the flap encompassing part of the stiffener, and the connector.

16. (canceled)

17. The sanitary product of claim 1, wherein the sensor is further configured and arranged for fluid detection.

18. The semi-manufactured sanitary product of claim 5, wherein the sensor is further configured and arranged for fluid detection.

19. The sanitary product according to claim 1, wherein the stiffener includes a stiffener width, in the short direction, in the range of 25-30 mm, and a stiffener length, in the long direction, in the range of 10-15 mm.
